(19) United States
(12) Patent Application Publication
(10) Pub. No.: US 2026/0325788 A1
(43) Pub. Date: Oct. 22, 2026

(54) FLEXIBLE CIRCUITS BASED CATHETERS

(71) Applicant: Nypro Inc., Clinton, MA (US)

(72) Inventor: Ramy Awad, Clinton, MA (US)

(73) Assignee: Nypro Inc., Clinton, MA (US)

(21) Appl. No.: 19/468,348

(22) Filed: Feb. 3, 2026

Related U.S. Application Data
(60) Provisional application No. 63/754,841, filed on Feb. 6, 2025.

(51) Int. Cl. A61M 25/00; A61B 5/287; A61B 18/14

(52) U.S. Cl. CPC A61M 25/0012; A61M 25/001; A61M 25/0045; A61B 5/287; A61B 18/1492; A61M 2207/10

(57) ABSTRACT

Catheters and methods of manufacturing the catheters based on flexible circuits are described. A catheter includes a component core, a flexible circuit wrapped around the component core, the flexible circuit including at least one or more conductive traces, an encapsulation layer wrapped around the flexible circuit, and one or more electrodes connected to one or more exposed circuit trace areas of the one or more conductive traces on the flexible circuit.

### CROSS-REFERENCE TO RELATED APPLICATIONS

This application claims priority to and the benefit of U.S. Provisional Patent Application No. 63/754,841, filed on Feb. 6, 2025, the entire contents of which are incorporated by reference herein for all purposes.

### TECHNICAL FIELD

This disclosure relates to catheters and in particular, use of flexible circuits to manufacture catheters.

### BACKGROUND

Catheters are medical devices that can be inserted into the body for diagnostic and therapeutic purposes, to perform a surgical procedure, deliver a therapy through the vasculature, remove fluids from the body, or provide fluids to the body, for example. There are a variety of catheters, including but not limited to, electrophysiology (EP) catheters, mapping electrode catheters, steerable catheters, guiding catheters, balloon catheters, and/or dialysis catheters. An EP catheter is a catheter that is a thin, flexible tube augmented with electronics such as sensors and electrodes. Manufacturing of catheters are labor intensive and inefficient. Moreover, these devices can have limited miniaturization capability, customization capability, and/or usability.

### SUMMARY

Disclosed herein are implementations of catheters and methods of manufacturing catheters based on flexible circuits including conductive traces and electronics. In implementations, a catheter includes a component core, a flexible circuit wrapped around the component core, the flexible circuit including at least one or more conductive traces, an encapsulation layer wrapped around the flexible circuit, and one or more electrodes connected to one or more exposed circuit trace areas of the one or more conductive traces on the flexible circuit.

### BRIEF DESCRIPTION OF THE DRAWINGS

The disclosure is best understood from the following detailed description when read in conjunction with the accompanying drawings and are incorporated into and thus constitute a part of this specification. It is emphasized that, according to common practice, the various features of the drawings are not to-scale. On the contrary, the dimensions of the various features are arbitrarily expanded or reduced for clarity.

FIG. 1 is a perspective view of a component in the manufacturing of a catheter in accordance with certain implementations.

FIG. 2 is a cross-sectional view of liner assembly in the manufacturing of a catheter in accordance with certain implementations.

FIG. 2A is another cross-sectional view of liner assembly in the manufacturing of a catheter in accordance with certain implementations.

FIG. 3 is a perspective view of a flexible circuit component in the manufacturing of a catheter in accordance with certain implementations.

FIG. 4 is a perspective view of another flexible circuit component in the manufacturing of a catheter in accordance with certain implementations.

FIG. 5 is a perspective cross-sectional view of flexible circuit component assembly in the manufacturing of a catheter in accordance with certain implementations.

FIG. 5A is another perspective cross-sectional view of flexible circuit component assembly in the manufacturing of a catheter in accordance with certain implementations.

FIG. 6 is a perspective cross-sectional view of encapsulation assembly in the manufacturing of a catheter in accordance with certain implementations.

FIG. 6A is another perspective cross-sectional view of encapsulation assembly in the manufacturing of a catheter in accordance with certain implementations.

FIG. 7 is a perspective cross-sectional view of ablating in the manufacturing of a catheter in accordance with certain implementations.

FIG. 7A is another perspective cross-sectional view of ablating in the manufacturing of a catheter in accordance with certain implementations.

FIG. 8 is a perspective cross-sectional view of electrode assembly in the manufacturing of a catheter in accordance with certain implementations.

FIG. 8A is another perspective cross-sectional view of electrode assembly in the manufacturing of a catheter in accordance with certain implementations.

FIG. 9 is another perspective cross-sectional view of electrode assembly in the manufacturing of a catheter in accordance with certain implementations.

FIG. 9A is another perspective cross-sectional view of electrode assembly in the manufacturing of a catheter in accordance with certain implementations.

FIG. 10 is a flow diagram of an example of a method for manufacturing a catheter using flexible circuit components.

FIG. 11 is a perspective view of a flexible circuit component in the manufacturing of a catheter in accordance with certain implementations.

FIG. 12 is a perspective cross-sectional view of the flexible circuit component of FIG. 11 in the manufacturing of a catheter in accordance with certain implementations.

FIG. 13 is a perspective view of another flexible circuit component in the manufacturing of a catheter in accordance with certain implementations.

FIG. 14 is a perspective cross-sectional view of encapsulation assembly and ablation in the manufacturing of a catheter in accordance with certain implementations.

FIG. 15 is a perspective cross-sectional view of electrode assembly in the manufacturing of a catheter in accordance with certain implementations.

### DETAILED DESCRIPTION

The figures and descriptions provided herein can be simplified to illustrate aspects of the described embodiments that are relevant for a clear understanding of the herein disclosed processes, devices, machines, manufactures, and/or compositions of matter, while eliminating for the purpose of clarity other aspects that can be found in typical similar devices, systems, compositions, and methods. Those of ordinary skill can thus recognize that other elements and/or steps can be desirable or necessary to implement the devices, systems, compositions, and methods described herein. However, because such elements and steps are well known in the art, and because they do not facilitate a better understanding of the disclosed embodiments, a discussion of such elements and steps may not be provided herein. However, the present disclosure is deemed to inherently include all such elements, variations, and modifications to the described aspects that would be known to those of ordinary skill in the pertinent art in light of the discussion herein.

Embodiments are provided throughout so that this disclosure is sufficiently thorough and fully conveys the scope of the disclosed embodiments to those who are skilled in the art. Numerous specific details are set forth, such as examples of specific aspects, devices, and methods, to provide a thorough understanding of embodiments of the present disclosure. Nevertheless, it will be apparent to those skilled in the art that certain specific disclosed details need not be employed, and that embodiments may be embodied in different forms. As such, the exemplary embodiments set forth should not be construed to limit the scope of the disclosure.

The terminology used herein is for the purpose of describing particular embodiments only and is not intended to be limiting. For example, as used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, steps, operations, elements, and/or components, but do not preclude the presence or addition of one or more other features, steps, operations, elements, components, and/or groups thereof.

The steps, processes, and operations described herein are thus not to be construed as necessarily requiring their respective performance in the particular order discussed or illustrated, unless specifically identified as a preferred or required order of performance. It is also to be understood that additional or alternative steps may be employed, in place of or in conjunction with the disclosed aspects.

Yet further, although the terms first, second, third, etc. may be used herein to describe various elements, steps, or aspects, these elements, steps, or aspects should not be limited by these terms. These terms may be only used to distinguish one element or aspect from another. Thus, terms such as “first,” “second,” and other numerical terms when used herein do not imply a sequence or order unless clearly indicated by the context. Thus, a first element, step, component, region, layer, or section discussed below could be termed a second element, step, component, region, layer, or section without departing from the teachings of the disclosure.

As used herein, the terminology “determine” and “identify,” or any variations thereof includes selecting, ascertaining, computing, looking up, receiving, determining, establishing, obtaining, or otherwise identifying or determining in any manner whatsoever using one or more of the devices and methods are shown and described herein.

As used herein, the terminology “example,” “the embodiment,” “implementation,” “aspect,” “feature,” or “element” indicates serving as an example, instance, or illustration. Unless expressly indicated, any example, embodiment, implementation, aspect, feature, or element is independent of each other example, embodiment, implementation, aspect, feature, or element and may be used in combination with any other example, embodiment, implementation, aspect, feature, or element.

As used herein, the terminology “or” is intended to mean an inclusive “or” rather than an exclusive “or.” That is unless specified otherwise, or clear from context, “X includes A or B” is intended to indicate any of the natural inclusive permutations. That is if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from the context to be directed to a singular form.

The non-limiting embodiments described herein are with respect to catheters or devices and methods for making and using the catheters or devices. The catheters or devices and method for making the catheters or devices may be modified for a variety of applications and uses while remaining within the spirit and scope of the claims. The embodiments and variations described herein, and/or shown in the drawings, are presented by way of example only and are not limiting as to the scope and spirit. The descriptions herein may be applicable to all embodiments of the device and the methods for making the devices.

Disclosed herein are implementations of catheters manufactured with flexible circuits, flexible hybrid circuits, and/or flexible trace circuits (collectively “flexible circuits”). In implementations, the flexible circuits can be formed by etching a pattern onto a conductive material clad flexible material, leaving behind conductive traces and/or pathways. In implementations, the flexible materials can be, but is not limited to, fabrics and/or polymers. In implementations, the flexible circuits can be formed by printing traces onto the flexible material.

In implementations, the flexible circuit can be wrapper around a liner, which in turn is wrapped around a removeable mandrel. In implementations, the liner can be wrapped around a memory metal core. In implementations, the liner can be wrapped around a core. In implementations, the core can be solid, hollow, partially hollow, single lumen, multi-lumen, multi-layered, multi-layered with multiple printed electronics layers therein, and/or combinations thereof. In implementations, the liner can be braided tubing to provide flexibility.

In implementations, the flexible circuit can be encapsulated in a thermoplastic elastomer, polyether block amide, and/or similar materials. In implementations, ablation can be used to expose portions of the flexible circuits. A variety of electrodes and/or sensors can then be connected to the exposed flexible circuits.

In implementations, the flexible circuit catheters can be steerable or fixed.

In implementations, the flexible circuit catheters can include multiple flexible circuit layers which can be connected using vias to electrodes, sensors, grounding, or combinations thereof.

In implementations, surface-mount technology (SMT) devices or components, such as but not limited to, capacitors, resistors, or connectors can be attached directly to the flexible circuits using a variety of materials such as but not limited to, conductive adhesives. Some of these adhesives can be removed through immersion in water or chemicals or through mechanical processes that preserve the integrity of the attached components. Such processes can enable the recovery of materials and components from these devices for reuse.

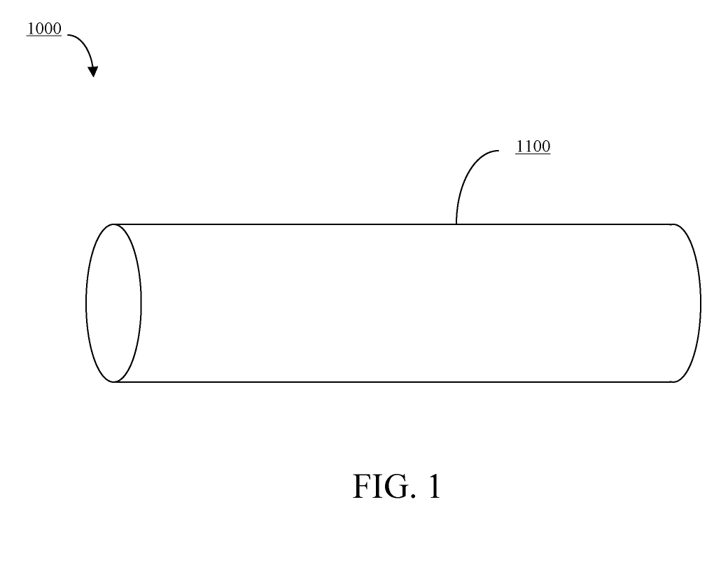

FIG. 1 is a perspective view of a core component 1100 in the manufacturing of a catheter 1000 in accordance with certain implementations. The catheter 1000 and the components therein can include other elements which may be desirable or necessary to implement the devices, systems, and methods described herein. However, because such elements and steps do not facilitate a better understanding of the disclosed embodiments, a discussion of such elements and steps may not be provided herein.

In implementations, the core component 1100 can be mandrel which can be removed after formation of the catheter 1000. The mandrel can be made of metal, plastic, and/or any material which provides a hard surface onto which to form the catheter and can be removed without impacting the catheter. In implementations, the mandrel can have ridges and/or other formations to maintain other components in place during the catheter formation process. In implementations, the mandrel can have indicators, such as raised points and/or bumps which align with a flexible circuit to indicate where the conductive traces are on the flexible circuit so that an ablation process can be done at the right places to expose the conductive traces. In implementations, the mandrel can have a tab, string, and/or other extension for removal of the mandrel from the formed catheter 1000.

In implementations, the core component 1100 can be memory metal or memory material, which can be shaped in a variety of shapes and forms depending on the catheter application or use.

In implementations, the core component 1100 can be a catheter core. The catheter core can be hollow, partially hollow, solid, single lumen, multi-lumen, multi-layered, multi-layered with multiple printed electronics layers therein, and/or combinations thereof. In implementations, the catheter core can be braided tubing, coiled tubing, and/or laminated with a laser cut hypotube to provide flexibility. In implementations, the lumens can carry fluid for irrigation, cooling and/or for balloon expansion. In implementations, the catheter core can include multiple layers which can be manufactured using co-extrusion, reflow techniques, and/or other processes. Printed electronics processes can be used to print electronics on each layer of the catheter core. The layers can be connected using vias, through holes, and/or other techniques. In implementations, thin electronics can be added to one or more layers and can be connected using vias, through holes, and/or other techniques.

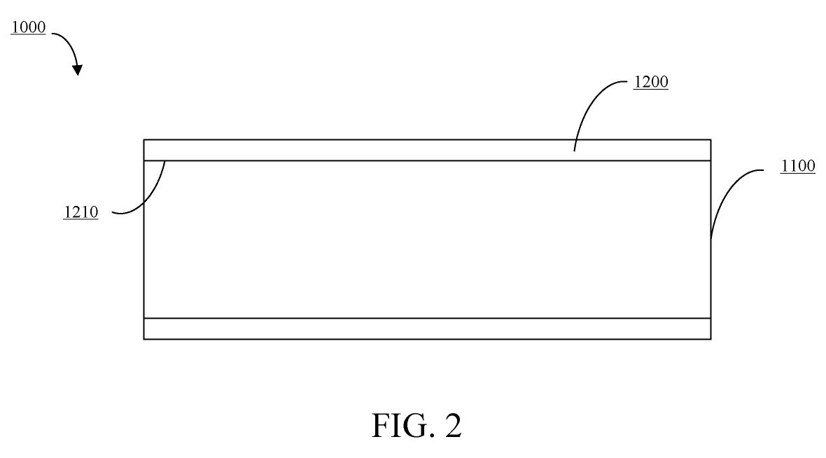

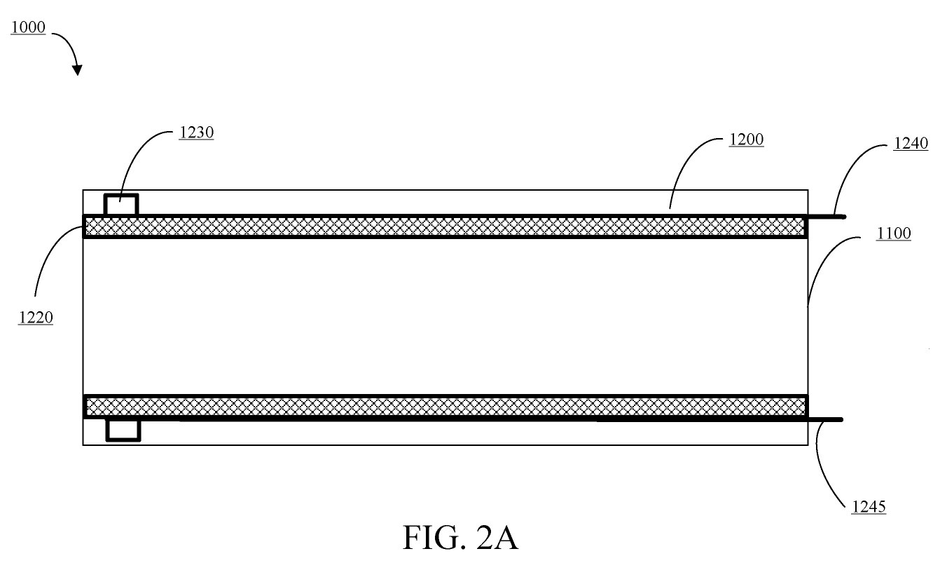

FIG. 2 is a cross-sectional view of liner assembly in the manufacturing of the catheter 1000 in accordance with certain implementations and FIG. 2A is another cross- sectional view of liner assembly in the manufacturing of a catheter in accordance with certain implementations. In implementations, a liner 1200 can wrap, encapsulate, envelope, and/or the like (collectively “wrap”) the core component 1100. In implementations, the liner 1200 can include an internal or core component facing surface 1210 which is lubricious. This enhances the removal of the core component 1100 when the core component 1100 is a mandrel. In implementations, the liner 1200 is optional when the core component 1100 is a polymer-based core, such as for example, a catheter core. In implementations, the liner 1200 can be used to ensure that there is no bonding between one layer an another.

In implementations, the liner 1200 can have braided tubing 1220 to provide flexibility.

In implementations, the liner 1200 can be made from, but is not limited to, a fluoropolymer, a polytetrafluoroethylene (PTFE), a fluorinated ethylene propylene (FEP), and/or an ethylene tetrafluoroethylene (ETFE).

In implementations, the catheter 1000 can be steerable or fixed. In an illustrative example, the liner 1200 can include an anchor ring 1230 connected to pull wires 1240 and 1245 which enable steering or guiding of the catheter 1000. In implementations, the pull wires 1240 and 1245 can have a variety of cross-sections including, but not limited to, flat, round, and/or shaped (where shaped can include a star shape, polygon-shape and the like). The liner 1200, the anchor ring 1230, and the pull wires 1240 and 1245 can be collectively referred to as a steerable sheath. In implementations, there can be multiple pull wires arranged in a way to steer the catheter in more than one plane. That is, the pull wires can be connected at different points on the anchor ring to provide greater a greater degree of freedom in controlling the catheter 1000. In implementations, additive manufacturing processes can be used to selectively add material at certain sections or portions of the liner 1200 to control the degree of steerability. That is, a thickness of the liner 1200 at these certain sections or portions can be greater than other sections or portions. In implementations, multiple anchor rings can be placed at a variety of sections along the liner 1200 to allow for steering along the length of the catheter 1000.

In implementations, the liner 1200 can include optical fiber for carrying laser beams for surgical operations, light for illumination, and/or combinations thereof, for example.

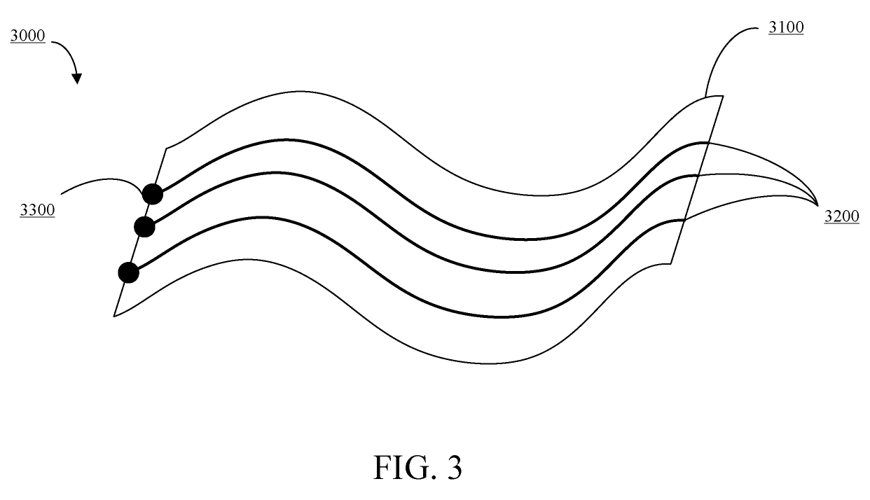

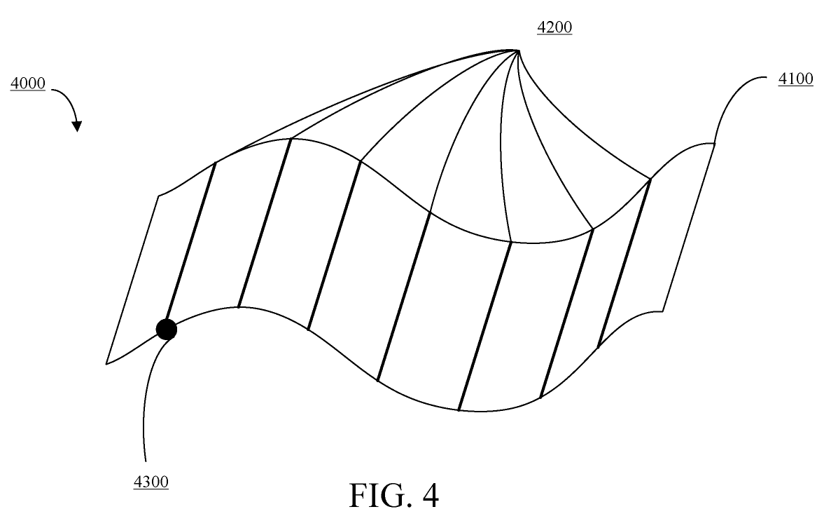

FIG. 3 is a perspective view of a flexible circuit component 3000 in the manufacturing of the catheter 1000 in accordance with certain implementations and FIG. 4 is a perspective view of another flexible circuit component 4000 component in the manufacturing of the catheter in accordance with certain implementations. In implementations, the flexible circuit component 3000 and the flexible circuit component 4000 can be formed by etching a pattern onto a conductive material clad flexible material and/or substrate 3100 and 4100, respectively, leaving behind conductive traces and/or pathways 3200 and conductive traces and/or pathways 4200, respectively. In implementations, the conductive material can be, but is not limited to, copper, gold, silver, and/or graphene. In implementations, the flexible material and/or substrate 3100 and 4100, respectively, can be, but is not limited to, fabrics, polymers, polyimide-based substrates, and/or thermosets. In implementations, the flexible circuit component 3000 and the flexible circuit component 4000 can be formed by printing the conductive traces and/or pathways 3200 and conductive traces and/or pathways 4200, respectively, onto the flexible material and/or substrate 3100 and 4100, respectively. In implementations, the conductive traces and/or pathways 3200 and the conductive traces and/or pathways 4200, respectively, can be terminated at one end and a remaining end can be connected to power.

In implementations, the flexible circuit component 3000 and the flexible circuit component 4000, respectively, can include one or more indicia, fiducial, marker, and/or clocking mechanism (indicia 3300 and indicia 4300, respectively) to indicate where the conductive traces and/or pathways 3200 and 4200 are located for later ablation and/or exposure and connection as described herein. In implementations, the indicia 3300 and 4300, respectively, can be raised protrusions relative to the external surface of the liner 1200. In implementations, the indicia 3300 and 4300, respectively, can be magnetic materials. In implementations, the indicia 3300 and 4300, respectively, can be sensitive to different frequencies, visible spectra, non-visible spectra, and/or combinations thereof.

In implementations, the flexible circuit component 3000 and the flexible circuit component 4000, respectively, can include one or more sensors such as, but not limited to, a temperature sensor, a force sensor, a pressure sensor, an electrical measurements sensor, and a navigation sensor.

In implementations, SMT components and other similar components can be attached to the conductive traces and/or pathways 3200 and 4200, respectively, using bonding and/or other techniques.

In implementations, the flexible circuit component 3000 and the flexible circuit component 4000, respectively, can include optical fiber for carrying laser beams for surgical operations, light for illumination, and/or combinations thereof, for example.

In implementations, the conductive traces and/or pathways 3200 and 4200, respectively, can be tested for continuity. Continuity can test for electron flow from one end of a conductive material to the other end. For example, a multimeter can be used for testing continuity.

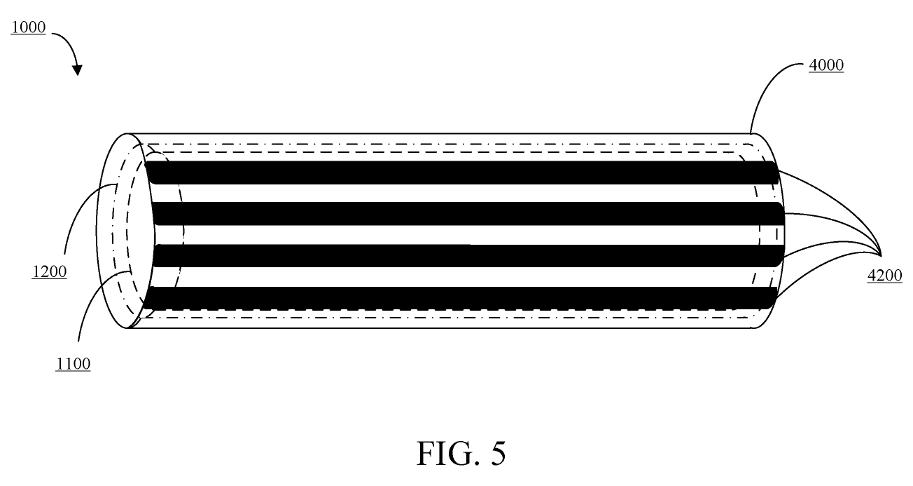

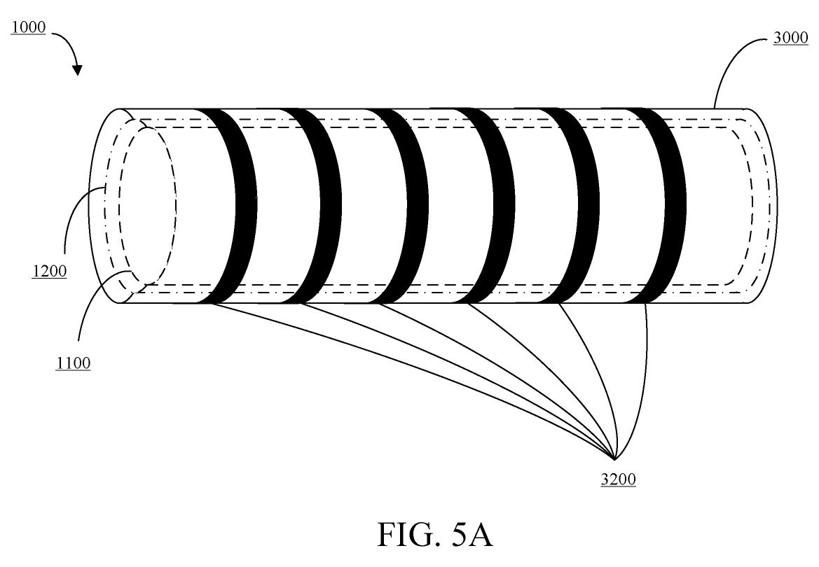

FIG. 5 is a perspective cross-sectional view of flexible circuit component assembly in the manufacturing of the catheter 1000 in accordance with certain implementations. In implementations, the flexible circuit component 4000 can be wrapped around the liner 1200 such that the conductive traces and/or pathways 4200 run parallel with an axis of the catheter 1000.

FIG. 5A is a perspective cross-sectional view of flexible circuit component assembly in the manufacturing of the catheter 1000 in accordance with certain implementations. In implementations, the flexible circuit component 3000 can be wrapped around the liner 1200 such that the traces or pathways 3200 run perpendicular to the axis of the catheter 1000.

In implementations, the flexible circuit component 3000 or the flexible circuit component 4000, respectively, can be wrapped around the liner 1200 such that the conductive traces and/or pathways 3200 and 4200, respectively, can face an outer surface of the liner 1200. In implementations, the flexible circuit component 3000 or the flexible circuit component 4000, respectively, can be wrapped around the liner 1200 such that the conductive traces and/or pathways 3200 and 4200, respectively, are on external surface of the flexible circuit component 3000 or the flexible circuit component 4000, respectively (e.g., non-facing surface with respect to the liner 1200).

In implementations, the two ends of the flexible circuit component 4000 or the flexible circuit component 3000, respectively, can be attached together. In implementations, the attachment can be done using a zipper-like or interlocking connection, via soldering, via bonding, via thermal bonding, via adhesive bonding and/or combinations thereof. In implementations, the flexible circuit component 4000 or the flexible circuit component 3000, respectively, can be rolled and secured through reflow (vertical or horizontal) or it can be film cast, dipped or over-molded.

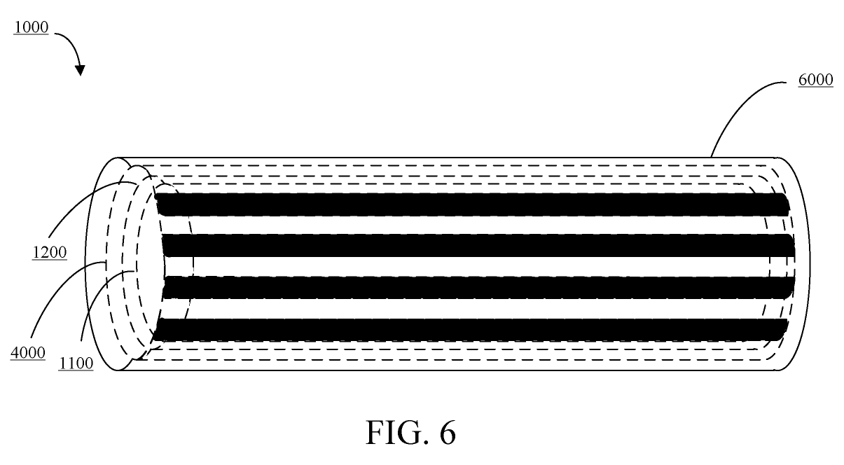

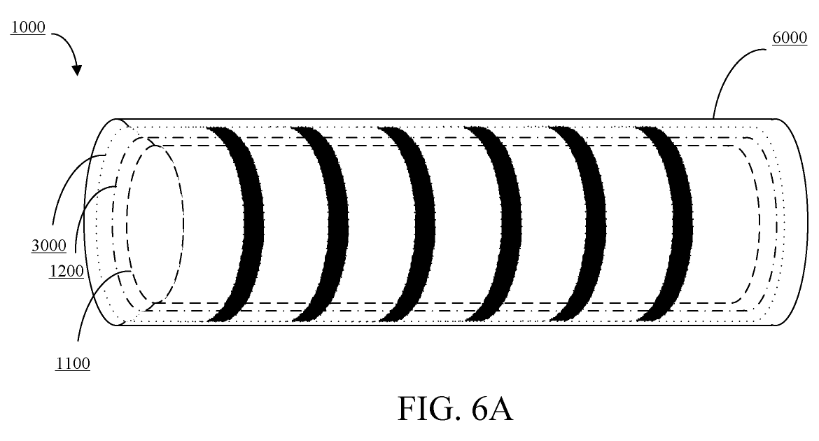

FIG. 6 is a perspective cross-sectional view of encapsulation assembly in the manufacturing of the catheter 1000 in accordance with certain implementations and FIG. 6A is another perspective cross-sectional view of encapsulation assembly in the manufacturing of the catheter 1000 in accordance with certain implementations. In implementations, the flexible circuit component 3000 or the flexible circuit component 4000, respectively, can be wrapped and/or laminated with an encapsulation layer or jacket 6000 such as, but not limited to, a thermoplastic elastomer, a polyether block amide, and/or similar materials. The encapsulation layer or jacket 6000 can insulate the conductive traces and/or pathways 3200 and 4200, respectively. In implementations, the encapsulation layer or jacket 6000 can be added using lamination processes or techniques, reflow processes or techniques, and/or similar processes or techniques. In these processes or techniques, annular or circular heaters traverse the catheter length at a specific speed and temperature and melt the polymer tube on the layer beneath via heat shrinking. The heat shrinking provides the force necessary, as it shrinks with the melting heat, to laminate a layer over another.

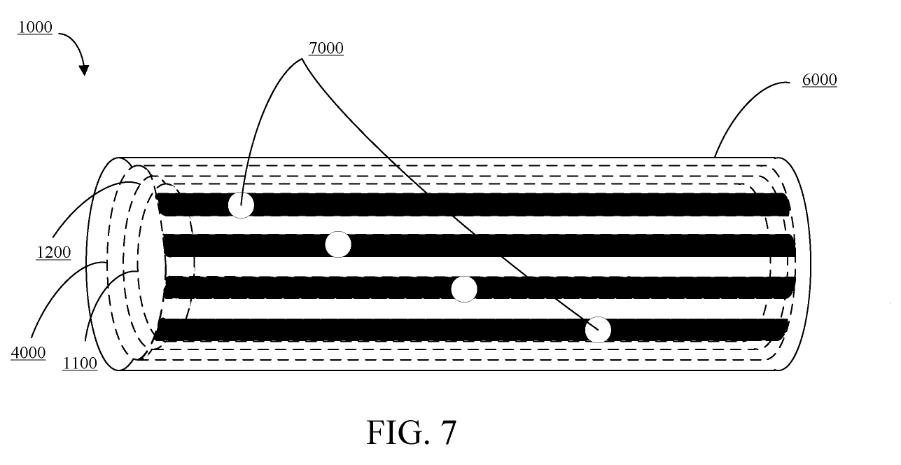

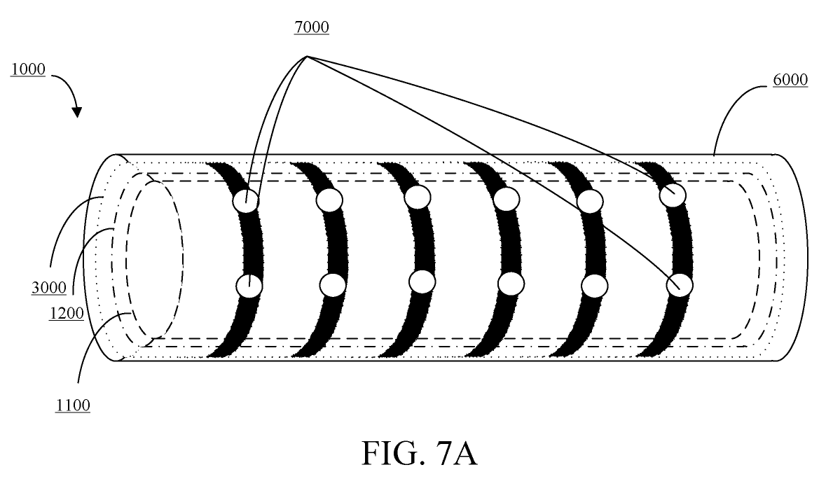

FIG. 7 is a perspective cross-sectional view of ablating in the manufacturing of the catheter 1000 in accordance with certain implementations and FIG. 7A is another perspective cross-sectional view of ablating in the manufacturing of the catheter in accordance with certain implementations. In implementations, laser ablation and/or similar techniques can be used to ablate the encapsulation layer or jacket 6000 and expose one or more conductive trace and/or pathway areas 7000 of the conductive traces and/or pathways 3200 and 4200, respectively. In implementations, one or more conductive trace and/or pathway areas 7000 can be exposed on each of the conductive traces and/or pathways 3200 and 4200, respectively.

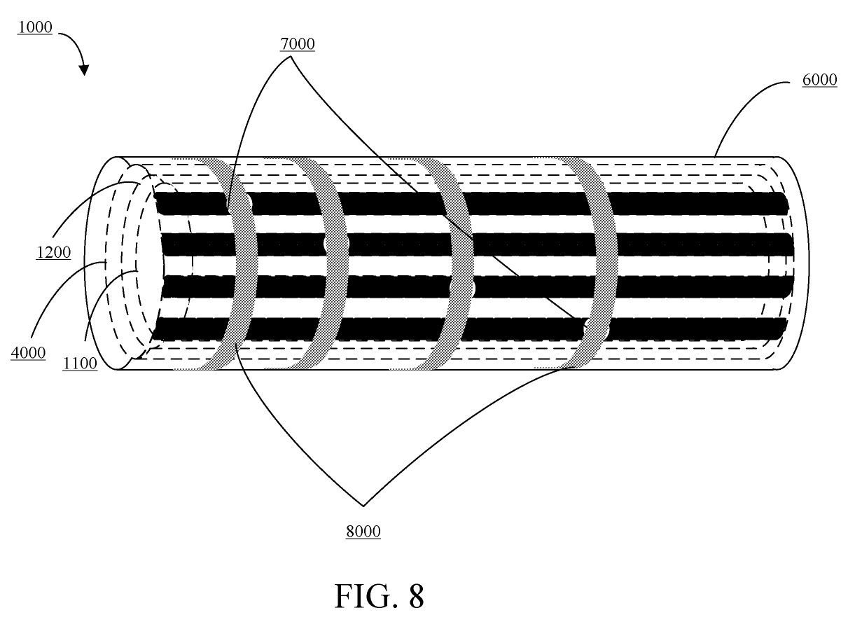

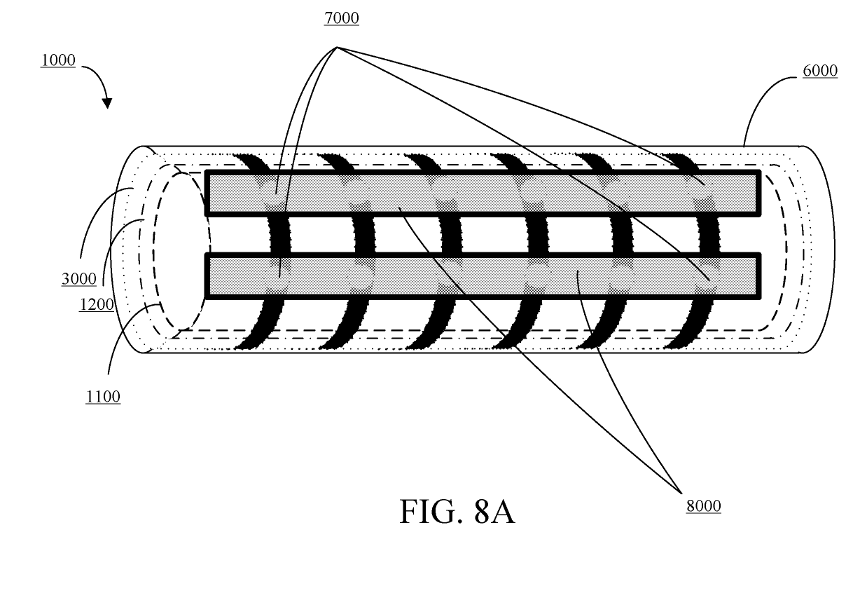

FIG. 8 is a perspective cross-sectional view of electrode assembly in the manufacturing of the catheter 1000 in accordance with certain implementations and FIG. 8A is another perspective cross-sectional view of electrode assembly in the manufacturing of the catheter 1000 in accordance with certain implementations. In implementations, one or more electrodes 8000 can be attached to the one or more conductive trace and/or pathway areas 7000 of the conductive traces and/or pathways 3200 and 4200, respectively. In implementations, lap welding techniques can be used to attach the one or more electrodes 8000 to the one or more conductive trace and/or pathway areas 7000. In implementations, the one or more electrodes 8000 are ring electrodes. In implementations, the one or more electrodes 8000 can be part of or integrated with a sensor, such as but not limited to, a temperature sensor, a force sensor, a pressure sensor, an electrical measurements sensor, and a navigation sensor.

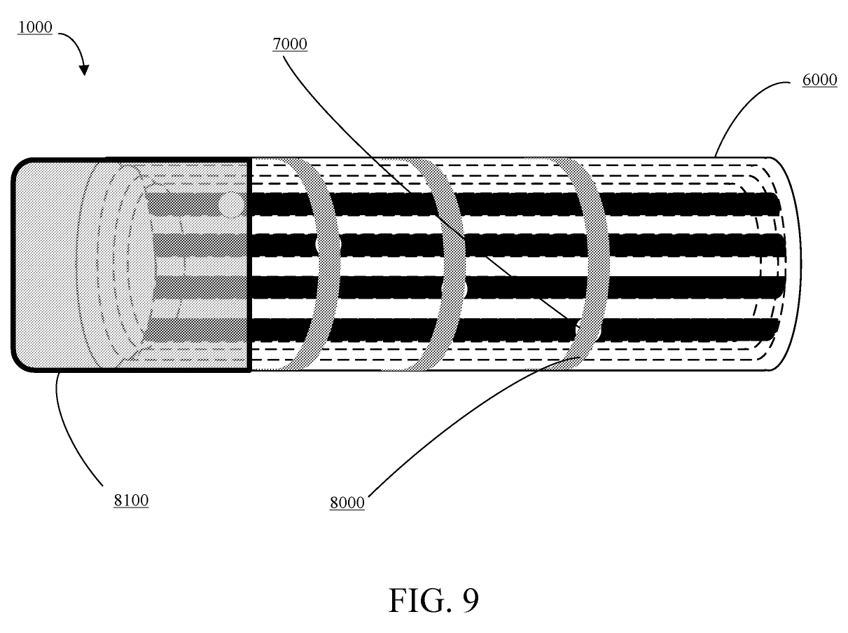

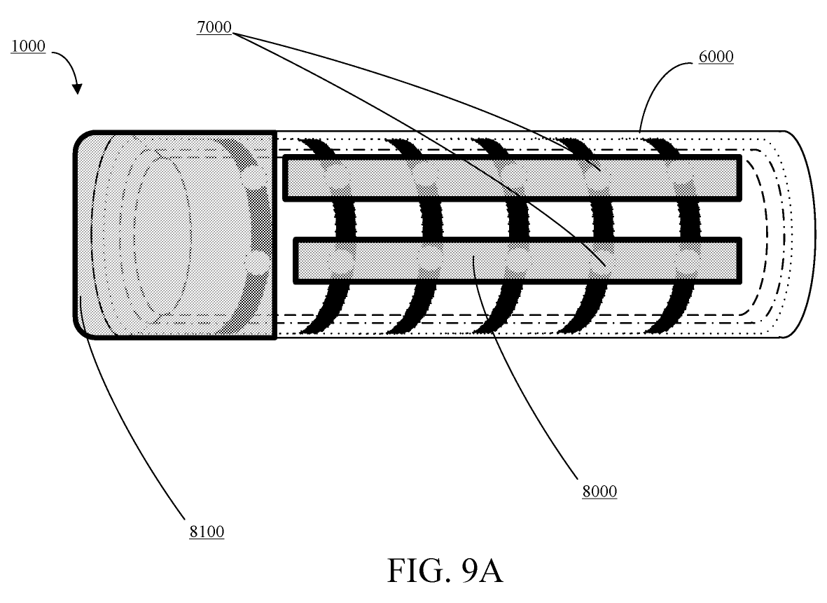

FIG. 9 is a perspective cross-sectional view of electrode assembly in the manufacturing of the catheter 1000 in accordance with certain implementations and FIG. 9A is another perspective cross-sectional view of electrode assembly in the manufacturing of the catheter 1000 in accordance with certain implementations. In implementations, one or more electrodes 8000 and a cap electrode 8100 can be attached to the one or more conductive trace and/or pathway areas 7000 of the conductive traces and/or pathways 3200 and 4200, respectively. In implementations, lap welding techniques can be used to attach the one or more electrodes 8000 and the cap electrode 8100 to the one or more conductive trace and/or pathway areas 7000. In implementations, the one or more electrodes 8000 are ring electrodes. In implementations, the one or more electrodes 8000 can be part of or integrated with a sensor, such as but not limited to, a temperature sensor, a force sensor, a pressure sensor, an electrical measurements sensor, and a navigation sensor. In implementations, the cap electrode 8100 can be used for mapping, denervation, and/or applications. In implementations, the one or more electrodes 8000 and the cap electrode 8100 can be used for forming and/or connecting a strain gauge. In implementations, the strain gauge can be used to measure the flex of the catheter 1000 as it touches or engages a cardiac vessel inside a ventricle or other internal area. As the catheter 1000 bends, the strain gauge can measure the change in resistance. For example, in an ablation catheter, increased pressure results in greater ablation. Accordingly, measurements from the strain gauge can be used to determine amount of force being applied. This can be used in a feedback loop to control pressure.

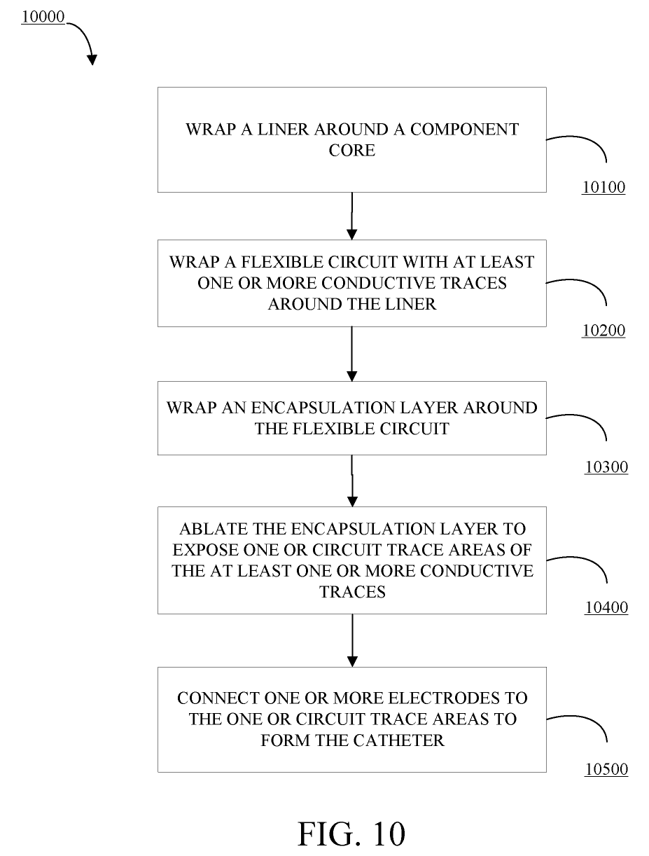

FIG. 10 is a flow diagram of an example of a method 10000 for manufacturing a catheter using a flexible circuit. The method 10000 can be used to make the catheters of FIGS. 1-9A. The method 10000 includes wrapping 10100 a liner around a component core; wrapping 10200 a flexible circuit with at least one or more conductive traces around the liner; wrapping 10300 an encapsulation layer around the flexible circuit; ablating 10400 the encapsulation layer to expose one or circuit trace areas of the at least one or more conductive traces; and connecting 10500 one or more electrodes to the one or circuit trace areas to form the catheter.

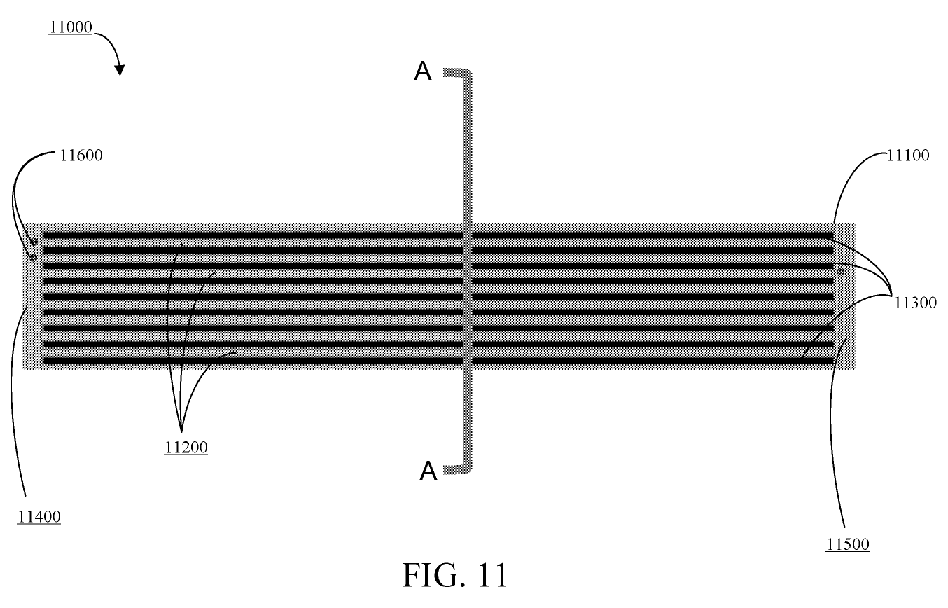

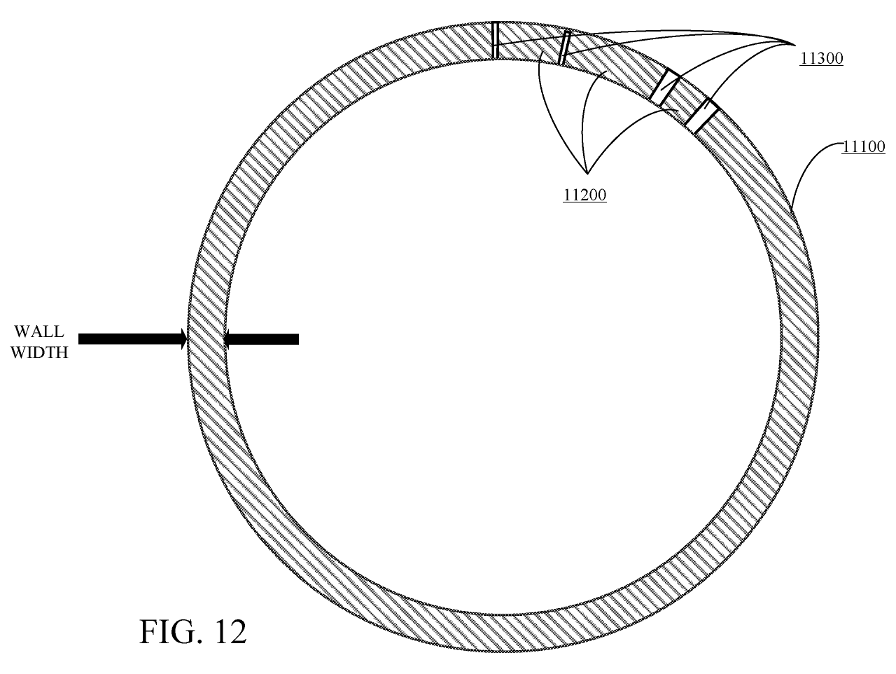

FIG. 11 is a perspective view of a flexible circuit component 11000 in the manufacturing of the catheter 1000 in accordance with certain implementations. FIG. 12 is a cross-sectional view of the flexible circuit component 11000 taken along A-A of FIG. 11 in accordance with certain implementations. In implementations, the flexible circuit component 11000 can be made by establishing one or more slits 11300 in a hypotube and/or conductive tube (collectively conductive tube 11100) leaving behind conductive traces and/or pathways 11200, a first anchor section 11400 and a second anchor section 11500. That is, the conductive traces and/or pathways 11200, the first anchor section 11400 and the second anchor section 11500 remain after the cutting of the slits. The first anchor section 11400 and the second anchor section 11500 maintains the integrity of the conductive tube 11100 after formation of the flexible circuit component 11000. In this instance, the flexible circuit component 11000 is the end product after the cutting of the slits in the conductive tube 11100. In implementations, the conductive tube 11100 can be, but is not limited to, copper, tantalum, nitinol, platinum, conductive metals, conductive precious metals, conductive exotic metals, metal alloys, precious metal alloys and/or combinations thereof. In implementations, a wall width of the conductive tube 11100 can be made to provide flexibility and maintain rigidity. In non-illustrative examples, the wall thicknesses for the conductive tube 11100 can range from 0.001" to 0.030" depending on the material of choice. The material of choice will play a huge role in the flexibility, malleability, ductility, and elasticity. In other words, the wall thickness specified for each application and size will depend on, but not limited to, the material type and/or grade of choice, the outer and inner diameter (i.e., the wall thickness), the slit geometry and shape, and other various handling and environmental factors.

In implementations, the core component 1100 can be used as described in FIG. 1 to indicate where the slits are to be established on the conductive tube 11100. In implementations, the establishment of the one or more slits 11300 can be done using, but not limited to, laser cutting, electrochemical grinding, machining, and/or combinations thereof.

In implementations, the spacing, shape, number, and/or size of slits 11300 and/or the conductive traces and/or pathways 11200 can be variable and can depend on the application for the catheter.

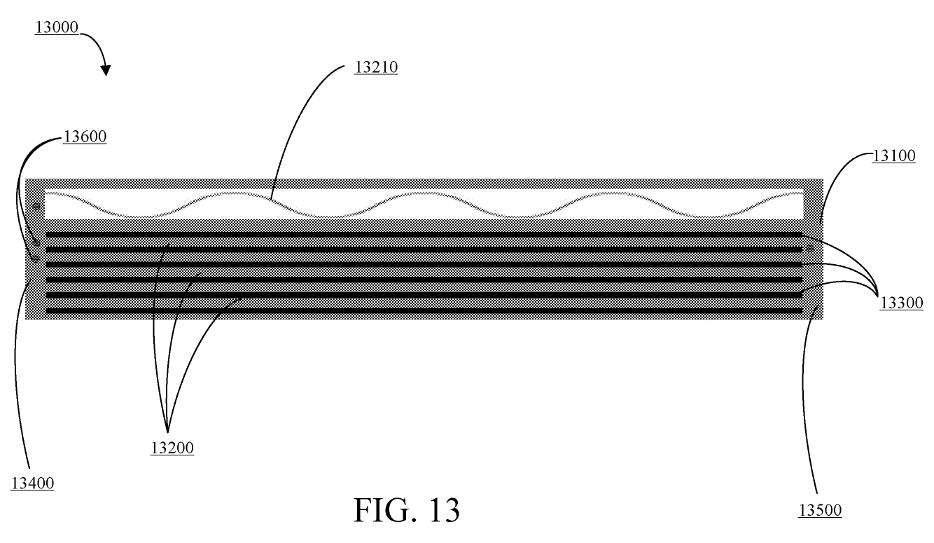

In implementations, a shape of a slit can be patterned to increase a bendability of the catheter. In a non-illustrative example, patterns (e.g., sinusoidal along a length) can be used to help as the catheter bends. The cutting pattern can be sinusoidal, which can result in a conductive trace being a wavy strip. FIG. 13 is a perspective view of a flexible circuit component 13000 in the manufacturing of the catheter 1000 in accordance with certain implementations. In implementations, the flexible circuit component 13000 can be made by establishing one or more slits 13300 in a conductive tube 13100 leaving behind conductive traces and/or pathways 13200 and a patterned trace and/or pathway 13210, a first anchor section 13400 and a second anchor section 13500.

In implementations, the flexible circuit component 11000 and/or 13000, respectively, can include one or more indicia, fiducial, marker, and/or clocking mechanism (indicia 11600 and indicia 13600, respectively), to indicate where the conductive traces and/or pathways 11200 and the conductive traces and/or pathways 13200 and the patterned trace and/or pathway 13210 are for later ablation and/or exposure and connection as described herein. In implementations, the indicia 11600 and 13600, respectively, can be on the first anchor section 11400 and the second anchor section 11500, and the first anchor section 13400 and the second anchor section 13500, respectively. In implementations, the indicia 11600 and 13600, respectively, can be raised protrusions relative to the external surface of the liner 1200 as shown in FIG. 1. In implementations, the indicia 11600 and 13600, respectively, can be magnetic materials which can be laser marked and/or engraved, pad printed, and/or manually marked. In implementations, the indicia 11600 and 13600, respectively, can be sensitive to different frequencies, visible spectra, non-visible spectra, and/or combinations thereof.

In implementations, the flexible circuit component 11000 and/or 13000 can be as described with respect to the flexible circuit component 3000 and flexible circuit component 4000.

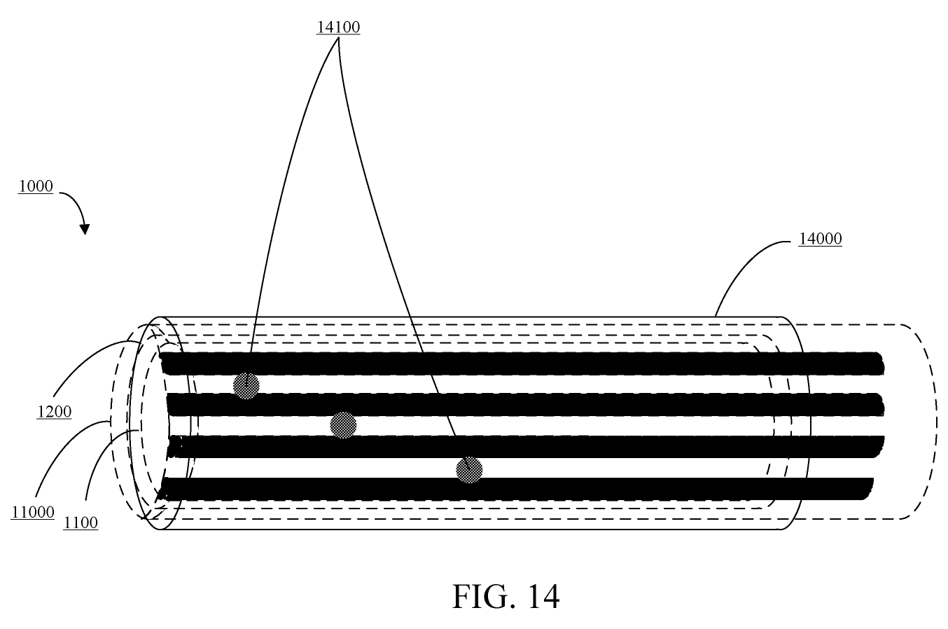

FIG. 14 is a perspective cross-sectional view of encapsulation assembly in the manufacturing of the catheter 1000 in accordance with certain implementations. In implementations, the flexible circuit component 11000 (also applicable to flexible circuit component 13000) can be wrapped and/or laminated with an encapsulation layer or jacket 14000 such as, but not limited to, Ethylene tetrafluoroethylene (ETFE), fluorinated ethylene propylene (FEP), PEBAX, a thermoplastic elastomer, a polyether block amide, a film cast polyimide, and/or similar materials. The encapsulation layer or jacket 6000 can insulate the conductive traces and/or pathways 11200. The encapsulation layer or jacket 6000 can insulate or not insulate the first anchor section 11400 and the second anchor section 11500 depending on manufacturing capabilities and/or efficiencies. In implementations, the encapsulation layer or jacket 6000 can be added using lamination processes or techniques, reflow processes or techniques, and/or similar processes or techniques. In these processes or techniques, annular or circular heaters traverse the catheter length at a specific speed and temperature and melt the polymer tube on the layer beneath via heat shrinking. The heat shrinking provides the force necessary, as it shrinks with the melting heat, to laminate a layer over another.

In implementations, laser ablation and/or similar techniques can be used to ablate the encapsulation layer or jacket 14000 and expose one or more conductive trace and/or pathway areas 14100 of one or more of the conductive traces and/or pathways 11200. In implementations, the laser ablation and/or similar techniques can be performed using the indicia 11600 on the first anchor section 11400 and the second anchor section 11500.

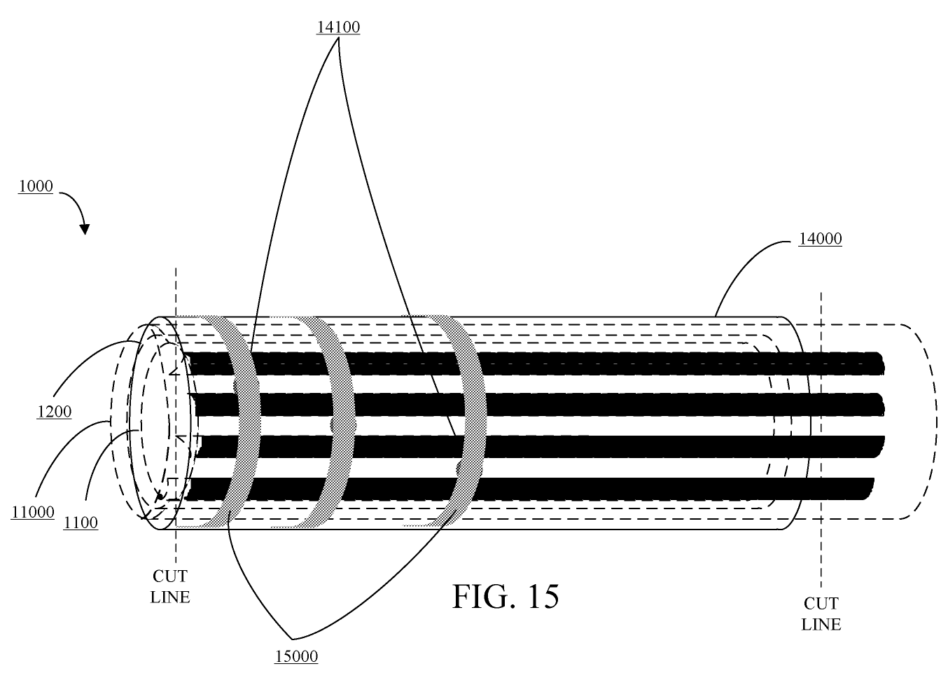

FIG. 15 is a perspective cross-sectional view of electrode assembly in the manufacturing of the catheter 1000 in accordance with certain implementations. In implementations, one or more electrodes 15000 can be attached to the one or more conductive trace and/or pathway areas 14100 of the conductive traces and/or pathways 11200. In implementations, lap welding techniques can be used to attach the one or more electrodes 15000 to the one or more conductive trace and/or pathway areas 14100. In implementations, the one or more electrodes 15000 are ring electrodes. In implementations, the one or more electrodes 15000 can be part of or integrated with a sensor, such as but not limited to, a temperature sensor, a force sensor, a pressure sensor, an electrical measurements sensor, and a navigation sensor.

In implementations, the uncovered conductive traces and/or pathways of the conductive traces and/or pathways 11200 can be terminated at one end and a remaining end can be connected to power. That is, the first anchor section 11400 and the second anchor section 11500 can be cut to reveal individual electrical signal and connect via wiring or flex circuit or hybrid circuit or any other common electrical connection.

In implementations, the catheter 1000 and/or portions thereof can be coated. In non-illustrative examples, the coating can be, but is not limited to, hydrophobic coatings, hydrophilic coatings, antimicrobial coatings, anticoagulant coatings, and/or combination thereof.

Described herein is a catheter which includes a component core, a flexible circuit wrapped around the component core, the flexible circuit including one or more conductive traces, an encapsulation layer wrapped around the flexible circuit, and one or more electrodes connected to one or more exposed circuit trace areas of the one or more conductive traces on the flexible circuit.

In implementations, the one or more conductive traces are etched patterns on a conductive material clad flexible material to form the flexible circuit. In implementations, the one or more conductive traces are printed on a flexible material to form the flexible circuit. In implementations, the one or more conductive traces are made by forming one or more slits on a conductive tube to form the flexible circuit. In implementations, the component core is a catheter core. In implementations, the catheter further includes a liner wrapped around the component core, wherein the flexible circuit is wrapped around the liner. In implementations, the liner is a steerable sheath to steer the catheter. In implementations, the flexible circuit includes one or more indicia to indicate a location of the one or more conductive traces. In implementations, the flexible circuit includes one or more sensors. In implementations, the catheter further includes one or more surface mount technology components attached to the one or more conductive traces. In implementations, the catheter further includes a cap electrode connected to the one or more exposed circuit trace areas.

Described herein is a method for manufacturing a catheter which includes wrapping a flexible circuit around a component core, the flexible circuit including one or more conductive traces, wrapping an encapsulation layer around the flexible circuit, ablating the encapsulation layer to expose one or circuit trace areas of the one or more conductive traces, and connecting one or more electrodes to the one or circuit trace areas to form the catheter.

In implementations, the method further includes wrapping a liner around a component core, wherein the flexible circuit is wrapped around the liner. In implementations, the method further includes removing the component core when the component core is a mandrel. In implementations, the method further includes etching patterns on a conductive material clad flexible material to form the one or more conductive traces and the flexible circuit. In implementations, the method further includes printing the one or more conductive traces on a flexible material to form the flexible circuit. In implementations, the flexible circuit includes one or more indicia to indicate a location of the one or more conductive traces for the ablating. In implementations, the method further includes connecting a cap electrode to the one or circuit trace areas.

Described herein is a catheter which includes a component core, a liner wrapped around the component core, a flexible circuit wrapped around the liner, the flexible circuit including one or more conductive traces formed by establishing slits in a conductive tube, an encapsulation layer wrapped around the flexible circuit, and one or more electrodes connected to one more exposed circuit trace areas of the one or more conductive traces of the flexible circuit.

In implementations, the component core is a mandrel.

The construction and arrangement of the methods as shown in the various exemplary embodiments are illustrative only. Although only a few embodiments have been described in detail in this disclosure, many modifications are possible (e.g., variations in sizes, dimensions, structures, shapes and proportions of the various elements, values of parameters, mounting arrangements, use of materials and components, colors, orientations, etc.). For example, the position of elements can be reversed or otherwise varied and the nature or number of discrete elements or positions can be altered or varied. Accordingly, all such modifications are intended to be included within the scope of the present disclosure. The order or sequence of any process or method steps can be varied or re-sequenced according to alternative embodiments. Other substitutions, modifications, changes, and omissions can be made in the design, operating conditions, and arrangement of the exemplary embodiments without departing from the scope of the present disclosure.

Although the figures can show a specific order of method steps, the order of the steps can differ from what is depicted. Also two or more steps can be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps, and decision steps.

While the disclosure has been described in connection with certain embodiments, it is to be understood that the disclosure is not to be limited to the disclosed embodiments but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A catheter comprising:
a component core;
a flexible circuit wrapped around the component core, the flexible circuit including one or more conductive traces;
an encapsulation layer wrapped around the flexible circuit; and
one or more electrodes connected to one or more exposed circuit trace areas of the one or more conductive traces on the flexible circuit.

2. The catheter of claim 1, wherein the one or more conductive traces are etched patterns on a conductive material clad flexible material to form the flexible circuit.

3. The catheter of claim 1, wherein the one or more conductive traces are printed on a flexible material to form the flexible circuit.

4. The catheter of claim 1, wherein the one or more conductive traces are made by forming one or more slits on a conductive tube to form the flexible circuit.

5. The catheter of claim 1, wherein the component core is a catheter core.

6. The catheter of claim 1, further comprising:
a liner wrapped around the component core, wherein the flexible circuit is wrapped around the liner.

7. The catheter of claim 6, wherein the liner is a steerable sheath to steer the catheter.

8. The catheter of claim 1, wherein the flexible circuit includes one or more indicia
to indicate a location of the one or more conductive traces.

9. The catheter of claim 8, wherein the flexible circuit includes one or more sensors.

10. The catheter of claim 8, further comprising:
one or more surface mount technology components attached to the one or more conductive traces.

11. The catheter of claim 8, further comprising:
a cap electrode connected to the one or more exposed circuit trace areas.

12. A method for manufacturing a catheter comprising:
wrapping a flexible circuit around a component core, the flexible circuit including one or more conductive traces;
wrapping an encapsulation layer around the flexible circuit;
ablating the encapsulation layer to expose one or circuit trace areas of the one or more conductive traces; and
connecting one or more electrodes to the one or circuit trace areas to form the catheter.

13. The method of claim 12, further comprising:
wrapping a liner around a component core, wherein the flexible circuit is wrapped around the liner.

14. The method of claim 13, further comprising:
removing the component core when the component core is a mandrel.

15. The method of claim 12, further comprising:
etching patterns on a conductive material clad flexible material to form the one or more conductive traces and the flexible circuit.

16. The method of claim 12, further comprising:
printing the one or more conductive traces on a flexible material to form the flexible circuit.

17. The method of claim 12, wherein the flexible circuit includes one or more indicia
to indicate a location of the one or more conductive traces for the ablating.

18. The method of claim 12, further comprising:
connecting a cap electrode to the one or circuit trace areas.

19. A catheter comprising:
a component core;
a liner wrapped around the component core;
a flexible circuit wrapped around the liner, the flexible circuit including one or more conductive traces formed by establishing slits in a conductive tube;
an encapsulation layer wrapped around the flexible circuit; and
one or more electrodes connected to one more exposed circuit trace areas of the one or more conductive traces of the flexible circuit.

20. The catheter of claim 19, wherein the component core is a mandrel.
